(19) United States
(12) Patent Application Publication
(10) Pub. No.: US 2011/0308519 A1
(43) Pub. Date: Dec. 22, 2011

(54) DRY POWDER FORMULATION COMPRISING AN ANTIMUSCARINIC DRUG

(75) Inventor: Francesca SCHIARETTI, Parma (IT)

(73) Assignee: Chiesi Farmaceutici S.p.A., Parma (IT)

(21) Appl. No.: 13/165,955

(22) Filed: Jun. 22, 2011

(30) Foreign Application Priority Data
Jun. 22, 2010 (EP) 10166903.4

(51) Int. Cl. A61M 15/00; A61K 31/439

(52) U.S. Cl. 128/203.15; 514/305

(57) ABSTRACT

The invention provides dry powder formulations suitable for the inhalatory administration by means of a dry powder inhaler, comprising an antimuscarinic drug as active ingredient. The invention also provides processes for the preparation of such a formulation and method for the prevention and/or treatment of a wide range of conditions including respiratory disorders by administering such a formulation.

### CROSS REFERENCES TO RELATED APPLICATIONS

This application claims priority to European Patent Application No. 10166903.4, filed on June 22, 2010, which is incorporated herein by reference in its entirety.

### BACKGROUND OF THE INVENTION

1. Field of the Invention

The present invention relates to dry powder formulations suitable for the inhalatory administration by means of a dry powder inhaler, comprising an antimuscarinic drug as active ingredient. The present invention also relates to processes for the preparation of such a formulation and to methods of using such a formulation for the prevention and/or treatment of a wide range of conditions including respiratory disorders.

2. Discussion of the Background

Airway obstruction characterizes a number of severe respiratory diseases including asthma and chronic obstructive pulmonary disease (COPD). Events leading to airway obstruction include oedema of airway walls, increased mucous production and inflammation.

Drugs for treating respiratory diseases such as asthma and COPD are currently administered through inhalation. One of the advantages of the inhalatory route over the systemic one is the possibility of delivering the drug directly at site of action, avoiding any systemic side-effects, thus providing a more rapid clinical response and a higher therapeutic ratio.

An important class of therapeutic agents used as bronchodilators is represented by the muscarinic receptor antagonist inhibitors belonging to the class of the quaternary ammonium salts, and in particular by the selective M3 receptor antagonists (hereinafter M3 antagonists). For example, M3 antagonists have been disclosed in WO 02/051841, WO 03/053966, and WO2008/012290, all of which are incorporated herein by reference in their entireties

Additional M3 receptor antagonists having high potency and long duration of action, that, once adsorbed, are degraded to inactive compounds which are deprived of any systemic side effects typical of muscarinic antagonists, are the object of a co-pending International Patent Application No. PCT/EP2009/008870, which is incorporated herein by reference in it is entirety. In particular, these latter compounds turned out to be particularly selective and endowed with a high potency. Therefore, said compounds may provide significant therapeutic benefit in the treatment of respiratory diseases such as asthma and COPD, when administered by inhalation.

Said drugs could be administered to the respiratory tract by inhalation in the form of dry powder by means of suitable inhalers known as dry powder inhalers (DPIs).

Thus, there remains a need for dry powder formulations suitable for the administration by inhalation of an antimuscarinic drug as an active ingredient by means of a dry powder inhaler. There also remains a need for processes for the preparation of such a formulation and for methods of using such a formulation for the prevention and/or treatment of a wide range of conditions including respiratory disorders.

### SUMMARY OF THE INVENTION

Accordingly, it is one object of the present invention to provide novel dry powder formulations which comprise an antimuscarinic drug.

It is another object of the present invention to provide novel dry powder formulations which are suitable for administration by inhalation of an antimuscarinic drug as an active ingredient.

It is another object of the present invention to provide novel dry powder formulations which are suitable for administration by inhalation of an antimuscarinic drug as an active ingredient and which exhibit good flowability, good uniformity of distribution of the active ingredient and adequate chemical and physical stability in the device before use.

It is another object of the present invention to provide novel dry powder formulations which are suitable for administration by inhalation of an antimuscarinic drug as an active ingredient and which give rise to a good respirable fraction as well as deliver an accurate therapeutically active dose of the active ingredient.

These and other objects, which will become apparent during the following detailed description, have been achieved by the inventors' discovery of dry powder formulations suitable for the inhalatory administration by means of a dry powder inhaler, comprising an aminoester derivative of formula (I), acting as muscarinic receptor antagonist.

Thus, in a first embodiment, the present invention provides dry powder formulations suitable for the inhalatory administration by means of a dry powder inhaler, comprising an aminoester derivative of formula (I), acting as muscarinic receptor antagonist.

The invention also provides processes for the preparation of such a formulation.

The present invention also provides methods for the prevention and/or treatment of a wide range of conditions including respiratory disorders, such as chronic obstructive pulmonary disease (COPD) and asthma, by administering such a formulation to a subject in need threreof.

The present invention also provides packages comprising an inhalable dry powder formulation and a dry powder inhaler.

### DETAILED DESCRIPTION OF THE PREFERRED EMBODIMENTS

The compositions of the present invention are pharmaceutical formulations in the form of inhalable dry powder, comprising, as active ingredient, micronized particles of a compound of general formula (I):

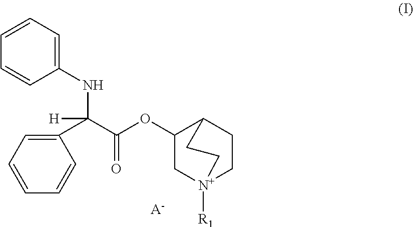

wherein:
R₁ is a group of formula (Y):
—(CH₂)ₚ—P—W

### (Y)

wherein:

p is 0 or an integer of 1 to 4;

P is absent or is selected from the group consisting of O, S, SO, SO₂ and CO;

W is selected from the group consisting of H, aryl, and heteroaryl, wherein aryl and heteroaryl are optionally substituted by one or more substituents selected from the group consisting of halogen atoms, —OH, —SH, —NO₂, —CN, —COOH and —NH₂;

A⁻ represents a physiologically acceptable anion; and

particles of a physiologically acceptable pharmacologically-inert solid carrier.

In the present invention, unless otherwise provided, the term “halogen” includes fluorine, chlorine, bromine, and iodine atoms.

The expression “aryl” refers to mono or bi- or tri-cyclic ring systems which have 6 to 20 ring atoms, preferably from 6 to 15 and wherein at least one ring is aromatic.

The expression “heteroaryl” refers to mono or bi-cyclic ring systems with 5 to 20 ring atoms, preferably from 5 to 15, in which at least one ring is aromatic and in which at least one ring atom is a heteroatom or heteroaromatic group (e.g. N, NH, S or O).

Examples of suitable aryl or heteroaryl monocyclic systems include, for instance, thiophene (thiophenyl), benzene (phenyl), pyrrole (pyrrolyl), pyrazole (pyrazolyl), imidazole (imidazolyl), isoxazole (isoxazolyl), oxazole (oxazolyl), isothiazole (isothiazolyl), thiazole (thiazolyl), pyridine (pyridinyl), imidazolidine (imidazolidinyl), furan (furanyl) radicals, and the like.

Examples of suitable aryl or heteroaryl bicyclic systems include naphthalene (naphthyl), biphenyl (biphenylyl), purine (purinyl), pteridine (pteridinyl), benzotriazole (benzotriazolyl), quinoline (quinolinyl), isoquinoline (isoquinolinyl), indole (indolyl), isoindole (isoindolyl) radicals and the like.

Advantageously, the physiologically acceptable anion A⁻ is selected from the group consisting of chloride, bromide, iodide, trifluoroacetate, formate, sulfate, phosphate, methanesulfonate, nitrate, maleate, acetate, citrate, fumarate, tartrate, oxalate, succinate, benzoate and p-toluenesulfonate.

In a first preferred embodiment, p is 1, P is absent, and W is H.

In a second preferred embodiment, p is 1, P is CO, and W is phenyl or thiophenyl.

In a third preferred embodiment, p is 2, P is O, and W is phenyl.

In a fourth preferred embodiment, p is 3, P is O, and W is phenyl.

The terms “active drug”, “active ingredient”, “active” and “active substance”, “active compound” and “therapeutic agent” are used synonymously.

The terms “muscarinic receptor antagonists”, “antimuscarinic drugs” and “anticholinergic drugs” are used synonymously.

The term “substantially pure” means a compound having a chemical or optical purity higher than 90% based on the weight of said compound, advantageously higher tan 95% w/w preferably higher than 98% w/w, more preferably higher than 99% w/w.

By “single therapeutically effective dose” it is meant the quantity of active ingredient administered at one time by inhalation upon actuation of the inhaler.

Said dose may be delivered in one or more actuations, preferably one actuation (shot) of the inhaler.

For “actuation” it is meant the release of active ingredient from the device by a single activation (e.g. mechanical or breath).

One aspect of the invention provides a pharmaceutical formulation, in the form of inhalable dry powder, comprising one or more compounds of formula (I) as active ingredients, and particles of a physiologically acceptable pharmacologically-inert solid carrier.

The invention provides a dry powder inhaler comprising the inhalable dry powder of the invention.

The invention also relates to the use of the inhalable dry powder formulation of the invention as a medicament.

A further aspect of the invention refers to the use of the inhalable dry powder of the invention for the prevention and/or treatment of an inflammatory or obstructive airways disease such as asthma or chronic obstructive pulmonary disease (COPD).

A still further aspect of the present invention refers to a method of preventing and/or treating an inflammatory or obstructive airways disease such as asthma or chronic obstructive pulmonary disease (COPD), which comprises administration by inhalation of an effective amount of the inhalable dry powder of the invention.

Finally the present invention is directed to a package comprising an inhalable dry powder formulation of the invention and a dry powder inhaler.

In general terms, the particle size of particles is quantified by measuring a characteristic equivalent sphere diameter, known as volume diameter, by laser diffraction.

The particle size can also be quantified by measuring the mass diameter by means of suitable known instruments such as, for instance the sieve analyser.

The volume diameter (VD) is related to the mass diameter (MD) by the density of the particles (assuming a size independent density for the particles).

In the following description, the particle size is expressed in terms of mass diameter (MD) and the particle size distribution is expressed in terms of: i) the mass median diameter (MMD) which corresponds to the diameter of 50 percent by weight or volume respectively, of the particles, and ii) the MD in microns of 10% and 90% of the particles, respectively.

The terms MMD and mean particle size are used synonymously.

The term “good flowability” refers to a formulation that is easily handled during the manufacturing process and is able to ensure an accurate and reproducible delivery of the therapeutically effective dose.

Flow characteristics can be evaluated by measuring the Carr's index; a Carr's index of less than 25 is usually taken to indicate good flow characteristics.

The expression “good homogeneity” refers to a formulation wherein, upon mixing, the content uniformity of the active ingredient, expressed as relative standard deviation (RSD), is less than 5%.

The expression “chemically stable” refers to a formulation that meets the requirements of the ICH Guideline Q1A referring to “Stability Testing of new Active Substances (and Medicinal Products)”.

The expression “physically stable in the device before use” refers to a formulation wherein the active particles do not substantially segregate and/or detach from the surface of the carrier particles during fabrication of the dry powder and in the delivery device before use.

The tendency to segregate can be evaluated according to Staniforth et al., J. Pharm. Pharmacol., 34,700-706, 1982, which is incorporated herein by reference in its entirety, and it is considered acceptable if the distribution of the active ingredient in the powder formulation after the test, expressed as relative standard deviation (RSD), does not change significantly with respect to that of the formulation before the test.

The expression “respirable fraction” refers to an index of the percentage of active particles which would reach the deep lungs in a patient. The respirable fraction, also termed fine particle fraction, is evaluated using a suitable in vitro apparatuses such as Multistage Cascade Impactor or Multi Stage Liquid Impinger (MLSI) according to procedures reported in common Pharmacopoeias. It is calculated by the ratio between the delivered dose and the fine particle mass (formerly fine particle dose). The delivered dose is calculated from the cumulative deposition in the apparatus, while the fine particle mass is calculated from the deposition on Stages 3 (S3) to filter (AF) corresponding to particles≦4.7 microns. A respirable fraction higher than 30% is an index of good inhalatory performances.

The expression “accurate therapeutically active dose of the active ingredient” refers to a formulation wherein the variation between the mean delivered daily dose and the mean emitted dose is equal to or less than 15%, preferably less than 10%.

According to specific embodiments of the invention, specific examples of compounds of formula (I), are reported in Table 1.

TABLE 1

| Compound | Chemical name and structure |
|---|---|
| C1 | (R)-1-(2-phenoxy-ethyl)-3-((R)-2-phenyl-2-phenylamino- acetoxy)-1-azonia-bicyclo[2.2.2]octane trifluoroacetate |
| | 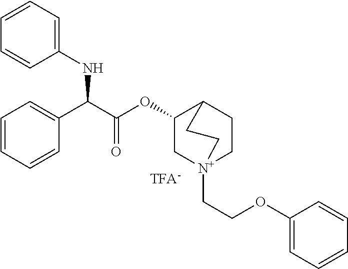 |
| C2 | (R)-1-(2-oxo-2-(thiophen-2-yl)ethyl)-3-(2-phenyl-2- (phenylamino)acetoxy)-1-azonia-bicyclo[2.2.2]octane trifluoroacetate |
| | 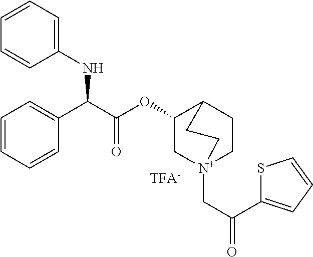 |
| C3 | (R)-1-(3-phenoxypropyl)-3-(2-phenyl-2-(phenylamino) acetoxy)-1-azonia-bicyclo[2.2.2]octane trifluoroacetate |
| | 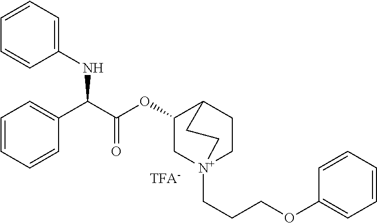 |
| C4 | (R)-1-methyl-3-(2-phenyl-2-(phenylamino)acetoxy)-1- azonia-bicyclo[2.2.2]octane trifluoroacetate |
| | 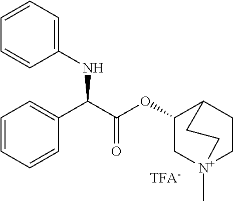 |
| C5 | (R)-1-(2-oxo-2-phenylethyl)-3-(2-phenyl-2-(phenylamino) acetoxy)-1-azonia-bicyclo[2.2.2]octane chloride |
| | 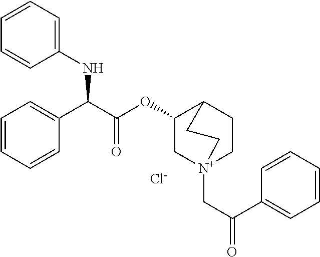 |

The compounds of general formula (I) show at least two chiral centers that are represented by the carbon atoms quoted with asterisks.

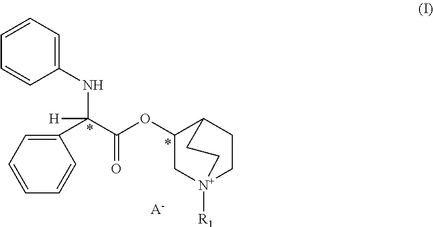

Therefore, the formula (I) also encompasses any of the optical stereoisomers, diastereoisomers and mixtures thereof, in any proportion.

The formulation of the present invention exhibits a good flowability, good uniformity of distribution of the active ingredient, and adequate chemical and physical stability in the device before use.

It also gives rise to a good respirable fraction and it is able of delivering an accurate therapeutically active dose of the active ingredient

The formulation of the present invention comprises the active ingredient in an amount such that, upon administration by inhalation from inhalers, the therapeutically effective single dose (hereinafter the single dose) is advantageously 5 μg to 2500 μg, more advantageously 10 μg to 2000 μg, preferably 15 to 1000 μg, more preferably 20 to 800 μg, even more preferably 25 to 600 μg.

The single dose will depend on the kind and the severity of the disease and the conditions (weight, sex, age) of the patient and shall be administered one or more times a day, preferably once or twice a day.

Without restricting the scope of the invention thereto, the pharmaceutical formulation may comprise any one of the compounds C1 to C5 in the form of the above indicated salts, such that the administered single dose is 10 μg to 600 μg, preferably 20 μg to 500 μg.

In a particular embodiment, the single dose may be 20 μg to 50 μg, while in other embodiments may be 40 μg to 100 μg or between 50 μg and 150 μg or 100 μg to 300 μg, or 300 to 500 μg.

In even more particular embodiments, the single dose may be of 20 or 25 or 50 or 100 or 200 or 500 μg.

If another salt is used, the single dose will vary on the basis of the different molecular weight of the counter ion.

The daily dose at which the pharmaceutical composition of the invention shall be 20 μg to 3000 μg, preferably 40 μg to 1000 μg and more preferably 50 pg to 500 μg. In one embodiment, the daily dose may be reached by a single or double administration.

In another preferred embodiment, the daily dose may be reached by a single administration and delivered in one actuation of the inhaler. In another preferred embodiment, the daily dose may be reached by a single administration and delivered in more actuations of the inhaler, preferably two. In another preferred embodiment, the daily dose may be reached by a double administration and delivered in one actuation of the inhaler. In another preferred embodiment, the daily dose may be reached by a double administration and delivered in more actuations of the inhaler, preferably two.

The particles of a compound of formula (I) in the formulation of the invention must be in a finely divided (micronized) form, i.e. their mass median diameter should generally be equal to or less than 10 μm, preferably less than 6 μm, more preferably 1 μm to 6 μm.

In certain embodiments of the invention, the particle size may fulfill the following requirements:

i) no more than 10% of the particles have a mass diameter lower than 0.8 μm;

ii) no more than 50% of particles have a mass diameter lower than 1.7 microns, preferably 1.8 to 2.5 microns; and

iii) at least 90% of the particles have a mass diameter lower than 6 microns.

The active ingredient may be produced in the desired particle size using known methods, e.g. milling, direct precipitation, spray-drying, freeze-drying or supercritical fluids.

The carrier particles may be made of any physiologically acceptable pharmacologically-inert material or combination of materials suitable for inhalatory use.

For example, the carrier particles may be composed of one or more materials selected from sugar alcohols; polyols, for example sorbitol, mannitol and xylitol, and crystalline sugars, including monosaccharides and disaccharides; inorganic salts such as sodium chloride and calcium carbonate; organic salts such as sodium lactate; and other organic compounds such as urea, polysaccharides, for example starch and its derivatives; oligosaccharides, for example cyclodextrins and dextrins.

Advantageously, the carrier particles are made of a crystalline sugar, for example, a monosaccharide such as glucose or arabinose, or a disaccharide such as maltose, saccharose, dextrose or lactose.

Preferably, the carrier particles are made of lactose, more preferably of α-lactose monohydrate.

In one embodiment the present invention, the powder formulation may be in form of agglomerated spheronized particles, also known as soft pellets, wherein the particles of a compound of general formula (I) and the particles of the carrier are both in a finely divided form, i.e. their mass median diameter is generally less than 10 micron, preferably from 1 to 6 micron.

Said kind of formulations may be prepared according to known methods.

Generally, the process comprises the steps of:

i) micronizing together the active ingredient and the carrier;

ii) subjecting the resulting co-micronized mixture to agglomeration and spheronisation.

Alternatively, the process comprises the following steps:

i) micronising separately the active ingredient and the carrier;

ii) mixing the micronized components; and

iii) subjecting the resulting mixture to agglomeration and spheronisation.

In another embodiment of the present invention, the formulation comprises coarse particles of a carrier together with the drug in the finely divided form, a type of formulation known as ordered mixture.

Advantageously, said coarse carrier particles have a mass diameter (MD) of at least 50 micron, more advantageously greater that 80 micron. Preferably, the MD is 90 microns to 500 microns. In certain embodiments of the invention, the MD may be 90 microns to 150 microns. In other embodiments, the MD may be 150 microns to 400 microns, with a MMD preferably greater than 175 micron, and more preferably the MD may be 210 microns to 355 microns.

The desired particle size may be obtained by sieving according to known methods. When their MD is comprised between 150 and 400 micron, the carrier coarse particles have preferably a relatively highly fissured surface, that is, on which there are clefts and valleys and other recessed regions, referred to herein collectively as fissures. The “relatively highly fissured” coarse particles can be defined in terms of fissure index or rugosity coefficient as described in WO 01/78695 and WO 01/78693, both of which are incorporated herein by reference in their entireties, and they can be characterized according to the description therein reported.

Said carrier coarse particles may also be characterized in terms of tapped density or total intrusion volume measured as reported in WO 01/78695, which is incorporated herein by reference in its entirety. The tapped density of the carrier coarse particles is advantageously less than 0.8 g/cm³, preferably between 0.8 and 0.5 g/cm³. The total intrusion volume is of at least 0.8 cm³, preferably at least 0.9 cm³.

When the formulation of the present invention is in form of the aforementioned ordered mixture, it may advantageously comprise an additive material able to promote the release of the active particles from the carrier particles on actuation of the inhaler device, and hence able of improving the respirable fraction. The additive material, which is preferably bound to the surface of the carrier coarse particles, is of a different material from the carrier particles. Advantageously, the additive material is an amino acid, preferably selected from the group consisting of leucine, isoleucine, lysine, valine, methionine, phenylalanine. The additive may be a salt of a derivative of an amino acid, for example aspartame or acesulfame K. In one embodiment of the invention the additive particles consist substantially of leucine, advantageously L-leucine. Alternatively, the additive material may include or consist of one or more water soluble surface active materials, for example lecithin, in particular soya lecithin.

In a particular embodiment of the invention, the additive material may include or consist of one or more lubricant selected from the group consisting of stearic acid and salts thereof such as magnesium stearate, sodium lauryl sulphate, sodium stearyl fumarate, stearyl alcohol, sucrose monopalmitate.

Other possible additive materials include talc, titanium dioxide, aluminium dioxide, and silicon dioxide.

Advantageously, the additive particles have a starting mean particle size of less than 35 microns. Preferably they have a mean particle size of not more than 15 microns, more preferably of not more than 10 microns.

The optimum amount of additive material shall depend on the chemical composition and other properties of the additive material. In general, the amount of additive shall be not more than 10% by weight, based on the total weight of the formulation. However, it is thought that for most additives the amount of additive material should be not more than 5% by weight, preferably not more than 2% by weight, or even not more than 1% by weight or not more than 0.5% by weight, based on the total weight of the formulation. In general, the amount of additive material is of at least 0.01% by weight based on the total weight of the formulation.

In one of the preferred embodiment of the invention, the additive material is magnesium stearate. The amount of magnesium stearate is generally 0.01 to 2% by weight, preferably 0.02 to 1%, more preferably 0.1% to 0.5% by weight, based on the total weight of the formulation.

In some embodiments, magnesium stearate may coat the surface of the carrier particles in such a way as that the extent of the molecular surface coating is at least of 5%, preferably more than 10%, more preferably more than 15%, even more preferably equal to or more than and 25%, and up to 90%, 95%, 98%, or even 99%. For very high extents of surface coating, i.e. higher than 60%, the coating may be achieved using the process described in the co-pending European Patent Application No. 10158951.3, which is incorporated herein by reference in is entirety.

The extent of molecular surface coating, which indicates the percentage of the total surface of the carrier particles coated by magnesium stearate, may be determined by water contact angle measurement as reported in WO 00/53157, which is incorporated herein by reference in is entirety. The extent to which the magnesium stearate coats the surface of the lactose particles can also be determined by scanning electron microscopy (SEM), a versatile analytical technique well known in the art.

Such microscopy may be equipped with an EDX analyzer (an Electron Dispersive X-ray analyzer), that can produce an image selective to certain types of atoms, for example magnesium atoms. In this manner it is possible to obtain a clear data set on the distribution of magnesium stearate on the surface of carrier particles.

SEM may alternatively combined with IR or Raman spectroscopy for determining the extent of coating, according to known procedures.

Another analytical technique that can advantageously be used is X-ray photoelectron spectroscopy (XPS), by which it has been possible to calculate both the extent of coating and the depth of the magnesium stearate film around the lactose particles. XPS measurements may be taken with commercially available instruments such as Axis-Ultra instrument from Kratos Analytical (Manchester UK), typically using monochromated Al Kα radiation according to known procedures.

The formulations of the present invention in the form of ordered mixture may also comprise fine particles of a physiologically acceptable pharmacologically- inert material with a mass median diameter (MMD) equal to or less than 15 microns, preferably equal to or less than 10 microns. The percentage of fine particles of physiologically acceptable pharmacologically-inert material is advantageously 0.1 to 40% by weight, based on the total amount of the formulation.

Preferably, the coarse particles and the fine particles are constituted of the same physiologically acceptable pharmacologically-inert material.

In a preferred embodiment of the present invention, in particular when the single dose of the active ingredient is equal to or less than 300 μg, preferably equal to or less than 200 μg, the formulation is in form of hard-pellets according to the teaching of WO 01/78693, which is incorporated herein by reference in its entirety.

Said formulation hence comprises:

i) particles of a compound of general formula (I) in a micronized form;

ii) a fraction of microparticles constituted of a mixture composed of particles of physiologically acceptable pharmacologically-inert material and particles of an additive material, said microparticles having a MMD equal to or less than 10 microns; and

iii) a fraction of particles of a physiologically acceptable pharmacologically-inert material having a highly fissured surface and a mass diameter (MD) of 150 microns to 400 microns, preferably 212 microns to 355 microns.

Advantageously, the fraction of microparticles is composed of 90 to 99.5% by weight of the physiologically acceptable pharmacologically-inert material and 0.5 to 10% by weight of the additive material, and the ratio between the fraction of microparticles and the fraction of coarse particles is 1:99 to 40:60% by weight, preferably 5:95 to 30:70% by weight, even more preferably 10:90 to 20:80% by weight.

Preferably the physiologically acceptable inert material is a-lactose monohydrate and the additive material is magnesium stearate.

In a more preferred embodiment, the fraction of microparticles is composed of 98 to 99% by weight of a-lactose monohydrate and 1 to 2% by weight of magnesium stearate and the ratio between the fraction of microparticles and the fraction of coarse particles made of a-lactose monohydrate is 10:90% by weight, respectively.

The amount of magnesium stearate in the final formulation is advantageously 0.01 to 1.0% by weight, preferably 0.05 to 0.5% by weight, more preferably 0.1 to 0.4% by weight, based on the total weight of the formulation.

The formulation in form of ordered mixture according to the present invention may be prepared according to known methods. Said methods comprise the step of mixing together the carrier coarse particles, the optional fine carrier particles and the additive particles, and finally adding the finely divided pharmaceutically active compound to the resulting mixture. The particularly preferred formulation according to the invention may be prepared according to the methods reported in WO 01/78693, which is incorporated herein by reference in its entirety.

Among the methods therein described, the formulation is preferably prepared according to a process which comprises the following steps:

a) preparing microparticles constituted of a mixture composed of particles made of physiologically acceptable pharmacologically-inert material and particles of the additive, the inert material and the additive being first-mixed together and then co-micronized;

b) mixing the microparticles of step a) with coarse particles of a physiologically acceptable pharmacologically-inert material such that microparticles adhere to the surface of the coarse particles;

c) adding by mixing the active particles in the micronized form to the particles of step b).

The co-micronization step may be carried out by known methods such as those reported in WO 02/00197, which is incorporated herein by reference in its entirety. Advantageously, said step is carrier out by milling, more preferably by using a jet mill according to the conditions reported in WO 01/78693, which is incorporated herein by reference in its entirety.

In a preferred embodiment, the microparticles of step a) obtained by co-micronization, are subjected to a conditioning step according to conditions disclosed in the co-pending European Patent Application No. 10160565.7, which is incorporated herein by reference in its entirety.

Advantageously, during the step a) the additive may be embedded in the formed microparticles, or alternatively, in the case of a lubricant such as magnesium stearate, the additive may coat the surface of the carrier particles in such a way as that the extent of molecular surface coating is at least of 5%, preferably more than 10%, more preferably more than 15%, even more preferably more than and 35%.

The extent of molecular surface coating indicates the percentage of the total surface of the carrier particles coated by magnesium stearate.

The presence of the additive material embedded in the microparticles may be detected according to known methods, for instance, by electron scanning microscope coupled to microcalorimetry.

Alternatively, as reported above, the extent of molecular surface coating may be determined by water contact angle measurement as reported in WO 00/53157, which is incorporated herein by reference in its entirety, or by other known tools.

The formulations of the present invention may further comprise other therapeutic agents useful for the prevention and/or treatment of a respiratory disease, e.g. beta₂-agonists such as salmeterol, milveterol and vilanterol; corticosteroids such as fluticasone propionate or furoate, flunisolide, mometasone furoate, rofleponide and ciclesonide; phosphodiesterase-4 (PDE4) inhibitors such as roflumilast and combinations thereof.

The dry powder formulation herein described, may be used in all customary dry powder inhalers such as unit dose or multidose inhalers. For example, the formulation of the invention may be filled in hard gelatine capsules, in turn loaded in a unit dose inhaler such as the Aerolizer™. Alternatively, the formulation as a powder may be filled in a multidose inhaler comprising a powder reservoir as described in WO 2004/012801, which is incorporated herein by reference in its entirety.

Administration of the formulation of the present invention may be indicated for prophylactic purposes or for symptomatic relief for a wide range of conditions including respiratory disorders such as chronic obstructive pulmonary disease (COPD) and asthma of all types. Other respiratory disorders for which the formulations of the invention may be beneficial are those characterized by obstruction of the peripheral airways as a result of inflammation and presence of mucus, such as chronic obstructive bronchiolitis, chronic bronchitis, emphysema, acute lung injury (ALI), cystic fibrosis, rhinitis, and adult or respiratory distress syndrome (ARDS).

In addition, the formulation of the present invention may be useful in treating smooth muscle disorders such as urinary incontinence and irritable bowel syndrome; skin diseases such as psoriasis; hyperhydrosis and sialorrhea; and gastrointestinal ulcers.

Other features of the invention will become apparent in the course of the following descriptions of exemplary embodiments which are given for illustration of the invention and are not intended to be limiting thereof.

### EXAMPLES

### Example 1

### Inhalable Dry Powder Formulation Comprising C1 (Formulation 1)

A powder formulation according to the invention has the composition reported in Table 2:

TABLE 2

| | Amounts | | |
|---|---|---|---|
| | Per shot of the | | |
| | inhaler | | Single dose |
| Components | mg | % | μg |
| Compound C1 | 0.2 | 1.0 | 200 |
| Alpha-lactose monohydrate 212-355 μm | 17.82 | 89.1 | |
| Co-micronized particles | 1.98 | 9.9 | |
| Total weight | 20 | | |

Compound C1 is micronized by known methods, to prepare the active substance in the form of particles having a typical particle size suitable for inhalation. The co-micronized particles are constituted of a mixture of α-lactose monohydrate and magnesium stearate in the ratio 98:2 w/w and are obtained by co-milling in a jet mill particles of α-lactose monohydrate having a mean particle size of less than 250 microns and magnesium stearate particles having a mean particle size of less than 35 microns. The final formulation is filled in hard gelatine capsules and loaded in the Aerolizer^{TM} inhaler.

The aerosol performances are evaluated using a Multi Stage Liquid Impinger (MSLI) according to the procedure described in European Pharmacopoeia 2^{nd} edition, 1995, part V.5.9.1, pages 15-17, which is incorporated herein by reference in its entirety.

### Example 2

### Inhalable Dry Powder Formulation Comprising C2 (Formulation 2).

A powder formulation with a similar composition of that of Example 1, but using compound C2, is prepared. The composition is reported in Table 3.

TABLE 3

| | Amounts | | |
|---|---|---|---|
| | Per shot of the | | |
| | inhaler | | Single dose |
| Components | mg | % | μg |
| Compound C2 | 0.2 | 1.0 | 200 |
| Alpha-lactose monohydrate 212-355 μm | 17.82 | 89.1 | |
| Co-micronized particles | 1.98 | 9.9 | |
| Total weight | 20 | | |

The formulation is filled in hard gelatine capsules and loaded in the Aerolizer^{TM} inhaler. The aerosol performances are determined as reported in Example 1.

### Example 3

### Inhalable Dry Powder Formulation Comprising C3 (Formulation 3).

A further powder formulation according to the invention is prepared with the composition reported in Table 4.

TABLE 4

| | Amounts | | |
|---|---|---|---|
| | Per shot of the | | |
| | inhaler | | Single dose |
| Components | mg | % | μg |
| Compound C3 | 0.100 | 1.0 | 100 |
| Alpha-lactose monohydrate 90-150 μm | 8.91 | 89.1 | |
| magnesium stearate | 0.99 | 9.9 | |
| Total weight | 10 | | |

The formulation is filled in the multidose dry powder inhaler described in WO 2004/012801, which is incorporated herein by reference in its entirety.

### Example 4

### Inhalable Dry Powder Formulation Comprising C4 (Formulation 4)/

A further powder formulation according to the invention is prepared with the composition reported in Table 5.

TABLE 5

| | Amounts | | |
|---|---|---|---|
| | Per shot of the | | |
| | inhaler | | Single dose |
| Components | mg | % | μg |
| Compound C4 | 0.200 | 2.0 | 200 |
| Alpha-lactose monohydrate 90-150 μm | 8.82 | 97.90 | |
| magnesium stearate | 0.98 | 0.10 | |
| Total weight | 10 | | |

The formulation is filled in the multidose dry powder inhaler described in WO 2004/012801, which is incorporated herein by reference in its entirety.

### Example 5

### Inhalable Dry Powder Formulation Comprising C5 (Formulation 5)

A powder formulation according to the invention was prepared as described in Example 1. Its composition reported in Table 6:

TABLE 6

| | Amounts | | |
|---|---|---|---|
| | Per shot of the | | |
| | inhaler | | Single dose |
| Components | mg | % | μg |
| Compound C5 | 0.2 | 1.0 | 200 |
| Alpha-lactose monohydrate 212-355 μm | 17.82 | 89.1 | |
| Co-micronized particles | 1.98 | 9.9 | |
| Total weight | 20 | | |

The final formulation was filled in hard gelatine capsules and loaded in the Aerolizer™ inhaler. The aerosol performances were evaluated as reported in Example 1.

The results, as a mean of two determinations, in terms of delivered dose (DD), fine particle mass (FPM), fine particle fraction (FPF), and mass median aerodynamic diameter (MMAD), are reported in Table 7.

TABLE 7

| DD (μg) | FPM (μg) | FPF (%) | MMAD (μm) |
|---|---|---|---|
| 61.1 | 39.8 | 65.0 | 2.2 |

The FPF turned out to be excellent, indicating that said kind of formulation is capable of providing good aerosol performance.

### Example 6

### Further Inhalable Dry Powder Formulations Comprising C5.

Powder formulations with a similar composition of that of Example 5 are prepared using different strengths of compound C5 and different percentages of co-micronized particles. The compositions are reported in Table 8 and 9.

TABLE 8

| | Strength | | | | | | | |
|---|---|---|---|---|---|---|---|---|
| | 10 μg/ | | 10 μg/ | | 100 μg/ | | 200 μg/ | |
| | 10 mg | | 10 mg | | 10 mg | | 10 mg | |
| α-lactose monohydrate | 8.991 | | 8.4915 | | 8.415 | | 8.82 | |
| 212-355 μm (mg) | | | | | | | | |
| α-lactose monohydrate | 89.9 | | 84.9 | | 84.0 | | 88.0 | |
| 212-355 μm (%) | | | | | | | | |
| Co-micronized | 0.999 | | 1.4985 | | 1.485 | | 0.98 | |
| particles (mg) | | | | | | | | |
| Co-micronized | 10 | | 15 | | 15 | | 10 | |
| particles (%) | | | | | | | | |
| Compound C5 (mg) | 0.010 | | 0.010 | | 0.100 | | 0.200 | |
| Compound C5 (%) | 0.1 | | 0.1 | | 1 | | 2.0 | |
| Total | 10 | mg | 10 | mg | 10 | mg | 10 | mg |

The formulation is filled in the multidose dry powder inhaler described in WO 2004/012801, which is incorporated herein by reference in its entirety.

TABLE 9

| | Strength | | | | | |
|---|---|---|---|---|---|---|
| | 20 μg/ | | 20 μg/ | | 200 μg/ | |
| | 20 mg | | 20 mg | | 20 mg | |
| α-lactose monohydrate | 17.982 | | 16.803 | | 16.83 | |
| 212-355 μm (mg) | | | | | | |
| α-lactose monohydrate | 89.9 | | 84.9 | | 84.0 | |
| 212-355 μm (%) | | | | | | |
| Co-micronized particles (mg) | 1.998 | | 2.997 | | 2.97 | |
| Co-micronized particles (%) | 10 | | 15 | | 15 | |
| Compound C5 (mg) | 0.020 | | 0.020 | | 0.200 | |
| Compound C5 (%) | 0.1 | | 0.1 | | 1.0 | |
| Total | 20 | mg | 20 | mg | 20 | mg |

The formulation is filled in hard gelatine capsules and loaded in the Aerolizer^{TM} inhaler.

### Example 7

### Assessment of the Bronchodilation Activity of the Compounds of the Invention

Airway reactivity was measured using barometric plethysmography (Buxco, USA). Male guinea pigs (500 to 600 g) were individually placed in plexiglass chambers.

After an acclimatisation period, the animals were exposed to nebulised saline for 1 minute to obtain airway baseline reading. This was followed by a 1 minute challenge with nebulised acetylcholine (Ach) −2.5 mg/mL.

After 60 minutes, 5 minute nebulisation of vehicle or the preferred compounds of the invention in the range 0.1 to 1 mM were applied and Ach challenge was then repeated after 2, 5, 24, 48 and 72 hours (h). Recording of pressure fluctuations in the chambers was taken for 5 minutes after each nebulisation and analyzed to calculate Enhanced Pause (Penh). Airway reactivity is expressed as percentage increase in Penh compared with Penh values from the nebulisation of vehicle.

Two hours after the end of nebulisation, the Ach-induced increase in Penh was dose-dependently inhibited by the compounds, with a maximal effect at around 1 mM.

As for the time-course of the effect, said compounds showed an increasing duration of action with increasing dose.

After inhalation of 0.1 mM of said compounds, the effect persisted significantly up to 24 hours.

The estimation of lung levels for said compounds achieved after nebulisation of a dose endowed with a submaximal bronchodilator activity at 2 hours after treatment reveals that the their retained dose in the target organ is less than 5 μg/kg. If an extrapolation of these results from guinea pig to human is made, it can be predicted that in patients the daily dose might be comprised between 20 and 500 μg.

Where a numerical limit or range is stated herein, the endpoints are included. Also, all values and subranges within a numerical limit or range are specifically included as if explicitly written out.

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1. An inhalable dry powder formulation comprising:
(a) micronized particles of at least one compound of formula (I): 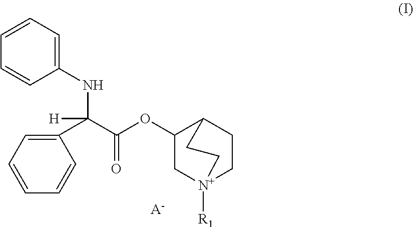
wherein:
R₁ is a group of formula (Y):
—(CH₂)ₚ—P—W
(Y)
wherein
p is 0 or an integer of 1 to 4;
P is absent or is selected from the group consisting of O, S, SO, SO₂, and CO;
W is selected from the group consisting of H, aryl, and heteroaryl, wherein said aryl and heteroaryl may be optionally substituted by one or more substituents selected from the group consisting of halogen atoms, —OH, —SH, —NO₂, —CN, —COOH and —NH₂;
A⁻ represents a physiologically acceptable anion; and
(b) particles of a physiologically acceptable pharmacologically-inert solid carrier.

2. An inhalable powder according to claim 1, wherein said physiologically acceptable anion A⁻ is selected from the group consisting of chloride, bromide, iodide, trifluoroacetate, formate, sulfate, phosphate, methanesulfonate, nitrate, maleate, acetate, citrate, fumarate, tartrate, oxalate, succinate, benzoate, and p-toluenesulfonate.

3. An inhalable powder according to claim 1, wherein said carrier comprises either (a) at least one crystalline sugar selected from the group consisting of glucose, arabinose, maltose, saccharose, dextrose, and lactose; or (b) at least one polyalcohol selected from the group consisting of mannitol, maltitol, lactitol, and sorbitol.

4. An inhalable powder according to claim 3, wherein said carrier comprises lactose.

5. An inhalable powder according to claim 3, wherein said carrier comprises α-lactose monohydrate.

6. An inhalable powder according to claim 1, wherein said carrier is in a form of finely divided particles having a mass median diameter (MMD) equal to or less than 10 microns.

7. An inhalable powder according to claim 1, wherein said carrier is in a form of coarse particles having a mass diameter of at least 50 microns.

8. An inhalable powder according to claim 7, wherein said coarse particles have a mass diameter of 150 microns to 400 microns.

9. An inhalable powder according to claim 1, wherein said carrier comprises a mixture of coarse particles having a mass diameter of 150 microns to 400 micron and finely divided particles having a MMD equal to or less than 10 microns.

10. An inhalable powder according to claim 6, further comprising one or more additive materials selected form the group consisting of an amino acid, a water soluble surface active agent, a lubricants, and a glidant.

11. An inhalable powder according to claim 10, wherein said additive material is a lubricant.

12. An inhalable powder according to claim 15, wherein the additive material is magnesium stearate.

13. An inhalable powder according to claim 17, wherein said magnesium stearate is present in an amount of 0.01 to 2% by weight, based on the total weight of the formulation.

14. An inhalable powder according to claim 15, wherein said magnesium stearate is present in an amount of 0.02 and 1% by weight, based on the total weight of the formulation.

15. A dry powder inhaler, comprising an inhalable dry powder formulation according to claim 1.

16. A method for the prevention and/or treatment of any disease wherein inhibition of the muscarinic receptor is required, said method comprising administering to a subject in need thereof an effective amount of an inhalable dry powder formulation according to claim 1.

17. A method according to claim 16, wherein said disease is respiratory disease.

18. A method according to claim 17, wherein said respiratory disease is asthma or chronic obstructive pulmonary disease.

19. A method according to claim 16, wherein said at least one compound of formula (I) is administered at a single dose of 5 μg to 2500 μg.

20. A method according to claim 16, wherein said at least one compound of formula (I) is administered at a single dose of 10 μg to 2000 μg.

21. A method according to claim 16, wherein said at least one compound of formula (I) is administered at a single dose of 15 μg to 1000 μg.

22. A method according to claim 16, wherein said at least one compound of formula (I) is administered at a single dose of 20 μg to 800 μg.

23. A method according to claim 16, wherein said at least one compound of formula (I) is administered at a single dose of 25 μg to 600 μg.

24. A package, comprising an inhalable dry powder formulation according to claim 1 and a dry powder inhaler.
